(19) United States
(12) Plant Patent Application Publication
(10) Pub. No.: US 2018/0124982 P1
(43) Pub. Date: May 3, 2018

(54) Petunia plant named 'PEHY0026'

(50) ;

(71) Applicant: SYNGENTA PARTICIPATIONS AG, Basel (CH)

(72) Inventor: Theodorus Cornelis Maria Van Kleinwee, Andijk (NL)

(73) Assignee: SYNGENTA PARTICIPATIONS AG, Basel (CH)

(21) Appl. No.: 15/731,594

(22) Filed: Jul. 6, 2017

(30) Foreign Application Priority Data
Oct. 31, 2016 (EM) 2016/2642

(51) Int. Cl. A01H 5/02

(52) U.S. Cl. PLT/356.23

(57) ABSTRACT

A new Petunia plant named ‘PEHY0026’ particularly distinguished by its medium sized, red flowers, mid early flowering and a semi-trailing plant habit.

Latin name of the genus and species of the plant claimed: Petunia hybrida.

Varietal denomination: ‘PEHY0026’.

### BACKGROUND OF THE NEW PLANT

The present invention comprises a new Petunia, botanically known as Petunia hybrida, and hereinafter referred to by the variety name ‘PEHY0026’.

‘PEHY0026’ is a product of a planned breeding program. The new cultivar has medium sized, red flowers, mid early flowering and a semi-trailing plant habit.

‘PEHY0026’ originated from a hybridization made in October 2011 in a controlled breeding environment in Enkhuizen, The Netherlands. The female parent was the proprietory line ‘N0187-1’ that has a comparable plant habit and vigor, is earlier, and has a less intense red flower color.

The male parent of ‘PEHY0026’ was the unpatented proprietary line ‘P0127’, a plant with comparable plant habit and vigor, later flowering and a less intensive flower color.

The resulting seeds were sown in January 2012. ‘PEHY0026’ was selected as one flowering plant within the progeny of the stated cross in May, 2012 in a controlled environment in Enkhuizen, The Netherlands.

The first act of asexual reproduction of ‘PEHY0026’ was accomplished when vegetative cuttings were propagated from the initial selection in June 2012 in a controlled environment in Angers, France.

### BRIEF SUMMARY OF INVENTION

Horticultural examination of plants grown from cuttings of the plant initiated in June 2012 and continuing thereafter, has demonstrated that the combination of characteristics as herein disclosed for ‘PEHY0026’ are firmly fixed and are retained through successive generations of asexual reproduction.

‘PEHY0026’ has not been observed under all possible environmental conditions. The phenotype may vary significantly with variations in environment such as temperature, light intensity, and day length.

A Plant Breeder's Right for this cultivar was applied for in the European Union on Oct. 31, 2016, No. 2016/2642. ‘PEHY0026’ has not been made publicly available prior to the effective filing date of this application, notwithstanding any disclosure that may have been made less than one year prior to the effective filing date of this application by the inventor or another who obtained ‘PEHY0026’ directly from the inventor.

The following traits have been repeatedly observed and are determined to be basic characteristics of the new variety. The combination of these characteristics distinguishes this Petunia as a new and distinct variety.

### BRIEF DESCRIPTION OF THE PHOTOGRAPHS

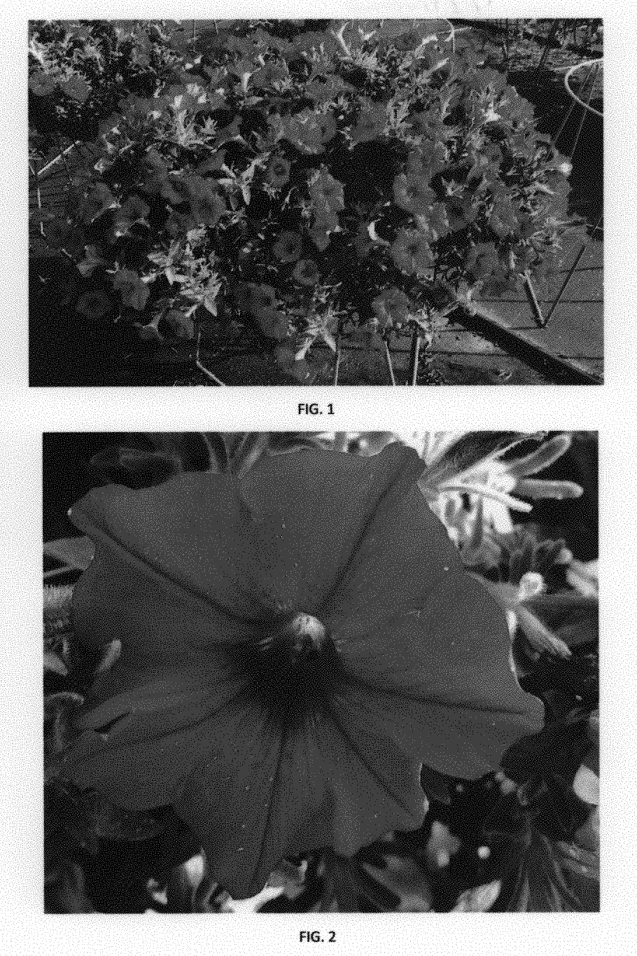

The accompanying photographic drawing shows typical flower and foliage characteristics of ‘PEHY0026’ with colors being as true as possible with an illustration of this type. The photographic drawing shows in FIG. 1. a flowering plant of the new variety and in FIG. 2. a close-up of the flowers.

### DETAILED BOTANICAL DESCRIPTION

The aforementioned photographs were taken in September 2016 from plants growing outside in Andijk, The Netherlands. These plants were approximately 22 weeks of age.

The plant descriptions and measurements were taken in October 2016 in Enkhuizen, Netherlands on 15 week old plants that were growing in full soil.

Color references are made to The Royal Horticultural Society Colour Chart (R.H.S.) 2001.

TABLE 1
DIFFERENCES BETWEEN THE NEW VARIETY ‘PEHY0026’
AND A SIMILAR VARIETY

| | | ‘PEHY0012’ (U.S. Pat. |
|---|---|---|
| | PEHY0026’ | Pat. No. PP28159) |
| Plant habit: | Semi-trailing | Semi-upright |
| Flower color: | Deeper red color | Red |
| Leaf shape: | Wider than ovate | Elliptic |

Plant:
   Form, growth and habit.—Semi-trailing plant habit.
   Plant height.—12 cm.
   Plant height (inflorescence included).—14 cm.
   Plant diameter (width).—35 cm.
Roots:
   Number of days to initiate and produce roots.—16 days at about 22 degrees C.
   Type.—Fine, fibrous, free branching.
   Color.—RHS N158D.
Foliage:
   Arrangement.—Alternate, simple; sometimes opposite, decussate.
   Immature leaf, color upper surface.—RHS 146B.
   Immature leaf, color lower surface.—RHS 146A.
   Mature leaf, color upper surface.—RHS 137A.
   Mature leaf, color lower surface.—RHS 146A.
   Length.—4 cm.
   Width.—2.8 cm.
   Shape.—Ovate.
   Base shape.—Shortly attenuate.
   Apex shape.—Acute.
   Margin.—Entire.
   Texture, upper surface.—Densely covered with short hair.
   Lower surface.—Densely covered with short hairs.
   Color of veins, upper surface.—RHS 144C.
   Color of veins, lower surface.—RHS 144C.
   Petiole color.—RHS 144C.
   Length.—0.3 cm.
   Diameter.—0.3 cm.
   Petiole texture.—densely covered with short hairs.
Stem:
   Quantity of main branches per plant.—4-5.
   Color of stem.—RHS 144A.
   Length of stem.—20-25 cm.
   Diameter.—0.3 cm.
   Length of internodes.—0.1-3 cm.
   Texture.—Dense, mostly glandular short hairs.
   Color of pedicel.—RHS 144A.
   Length of pedicel.—1.5-3.0 cm.
   Diameter of pedicel.—0.2 cm.
   Texture of pedicel.—Densely covered with short hairs.
Inflorescence:
   Type.—Flowers solitary in upper leaf axis.
   Floret type.—Funnel-shaped; 5 lobed petals; fused at the base.
   Blooming habit.—Rather late flowering in greenhouse, flowering throughout the summer months.
   Quantity of inflorescences per plant.—Approximately 15.
   Lastingness of individual blooms on the plant.—About 7 days.
   Fragrance.—Rather strong smell.
Bud (just when opening):
   Color.—RHS N79A.
   Length.—3.5 cm.
   Width.—0.8 cm.
   Shape.—Oblong.
Immature inflorescence:
   Flower horizontal diameter.—5 cm.
   Color petals, upper surface.—RHS 45A with veins RHS 53A.
   Color petals, lower surface.—RHS 54C with veins RHS 79D.
Mature flower:
   Flower horizontal diameter.—6 cm.
   Flower height (vertical).—4.5 cm.
   Color upper surface.—RHS 45A with veins RHS 53A.
   Color lower surface.—RHS 54C with veins RHS 79D.
   All petals.—
   Apex shape.—Rounded to slightly Acuminate.
   Margin.—Entire.
   Waviness of margin.—Medium.
   Petal lobation.—Little.
   Petal texture, upper surface.—Papillose, glabrous.
   Petal texture, lower surface.—Some glandular hairs, more dense on veins.
   Corolla tube color outside.—RHS 79A.
   Corolla tube color inside.—Longitudinal striped; RHS 53A and RHS 158D at base some RHS 157B coloration.
   Corolla tube length.—3 cm.
   Corolla tube texture, outside.—Rather dense and short glandular hairs.
   Corolla tube texture, inside.—Glabrous.
Calyx:
   Color of sepal, upper surface.—RHS 146A.
   Color of sepal, lower surface.—RHS 146B.
   Length.—1.7 cm.
   Width.—0.3 cm.
   Shape.—Ensiform.
   Apex shape.—Acute.
   Base.—Fused.
   Margins.—Entire.
   Texture, upper surface.—Densely covered with short glandular hairs.
   Lower surface.—Densely covered with short glandular hairs.
Reproductive organs:
   Pistil.—1.
   Length.—2.3 cm.
   Style color.—RHS 145C.
   Style length.—1.5 cm.
   Stigma color.—RHS 145A.
   Stamens.—
   Quantity per flower.—5.
   Color of filaments.—RHS 157C.
   Length filaments.—2 cm.
   Anther color.—RHS 159C.
   Anther length.—0.15 cm.
   Anther shape.—Elliptical.
   Color of pollen.—RHS 159D.
   Pollen amount.—Abundant.
   Fertility/seed set.—Has not been observed on this hybrid.
   Disease/pest resistance.—Has not been observed on this hybrid.

What is claimed is:

1. A new and distinct variety of Petunia plant named ‘PEHY0026’ substantially as illustrated and described herein.
